SEQUENCE LISTING (4 sequences)

SEQ ID NO: 1 - DNA, 20 bp, human
gtttacaggcttcatcaagg

SEQ ID NO: 2 - DNA, 20 bp, human
catgatggcatctgtcacag

SEQ ID NO: 3 - DNA, 20 bp, human
tgtccctgtatgcctctggt

SEQ ID NO: 4 - DNA, 19 bp, human
gatgtcacgcacgatttcc

(19) United States
(12) Patent Application Publication
(10) Pub. No.: US 2010/0267014 A1
(43) Pub. Date: Oct. 21, 2010

(54) Method for Prognosis of a Response to a Treatment

(75) Inventors: Myew-Ling Toh, Lyon (FR); Pierre Miossec, Bron (FR); Jean-Luc Blond, Ecully (FR); Bruno Mougin, Lyon (FR)

Correspondence Address: OLIFF & BERRIDGE, PLC, P.O. BOX 320850, ALEXANDRIA, VA, 22320-4850, US

(73) Assignee: HOSPICES CIVILS DE LYON, Lyon (FR)

(21) Appl. No.: 11/883,527

(22) PCT Filed: Feb. 28, 2006
(86) PCT No.: PCT/FR06/50175

§ 371 Date: Aug. 1, 2007

(30) Foreign Application Priority Data
Mar. 1, 2005 (FR) 0550540

(51) Int. Cl. C12Q 1/68; C07H 21/04

(52) U.S. Cl. 435/6; 536/24.31

(57) ABSTRACT

The present invention concerns an in vitro method for determining, from a biological sample, the response to a patient suffering from rheumatoid arthritis to a treatment directed against a cytokine involved in the inflammatory process of the disease, characterized in that it consists in measuring the expression of the gene encoding synoviolin.

The present invention relates to rheumatoid arthritis, and in particular to a method for determining the response of a patient suffering from rheumatoid arthritis to a treatment directed against a cytokine involved in the inflammatory process of the disease.

Rheumatoid arthritis (RA) is a chronic condition which is characterized by the inflammation and deformation of several joints with, in addition, a risk of extra-articular complications. Knowledge concerning the role of cytokines in cell-cell interactions has led to the reasonable development of treatments with anticytokine agents.

The seriousness of the condition varies from one individual to the other, but it is associated, long-term, with an increase in morbidity and mortality. Symptomatic treatment calls upon nonsteroidal anti-inflammatories and, optionally, on corticosteroids. Currently, methotrexate appears to be the treatment of reference. Treatments that inhibit proinflammatory cytokines are also proposed for the basic treatment of rheumatoid arthritis. In this respect, mention may be made of etanercept and Infliximab®, which are two inhibitors directed against TNF (tumor necrosis factor), a cytokine involved in the inflammatory process of rheumatoid arthritis. Such molecules are described as anti-TNF. More generally, the factor TNF-alpha has emerged as a main therapeutic target based on clinical studies with biological inhibitors such as monoclonal antibodies or soluble receptors. In this respect, Infliximab® is prescribed for reducing the inflammation, but also for slowing down the evolution of rheumatoid arthritis when other medicaments are insufficient. However, not all patients respond comparably to an Infliximab® treatment. Thus, X-rays of joints of patients suffering from rheumatoid arthritis and treated with Infliximab®, taken after one year, have revealed that, while a considerable number of patients benefited from an improvement, a smaller number had experienced joint deterioration. In a comparable manner, not all patients respond comparably to an etanercept treatment.

It is therefore important, from a clinical point of view, to determine, before any prescription, whether the patient will or will not respond to the treatment proposed by the physician.

At the current time, no method exists for determining, prior to a treatment with an anti-TNF, what the response of a patient taken individually might be.

The present invention proposes to solve the drawbacks of the prior art by providing a novel biological tool for improving the treatment of a patient against rheumatoid arthritis. The present invention in fact makes it possible to determine the response of a patient suffering from rheumatoid arthritis to a treatment such as Infliximab®. The present invention is also very relevant for monitoring the response of a patient subject to a treatment such as Infliximab®.

Surprisingly, the inventors have demonstrated that the response of a patient to such a treatment can be determined by analyzing the expression of the gene encoding synoviolin, in particular in the peripheral blood. The inventors have also demonstrated that the monitoring of the response of a patient to a treatment such as Infliximab® can be carried out by monitoring the expression of the gene encoding synoviolin.

To this effect, the invention relates to an in vitro method for determining, from a biological sample, the response of a patient suffering from rheumatoid arthritis to a treatment directed against a cytokine involved in the inflammatory process of the disease or for monitoring the response of a patient suffering from rheumatoid arthritis to a treatment directed against a cytokine involved in the inflammatory process of the disease, over time, characterized in that the expression of the gene encoding synoviolin is measured.

Preferably, the treatment is directed against the TNF-alpha cytokine. In this respect, mention may be made of a treatment that blocks the action of TNF, such as in particular Infliximab®, etanercept and adalimumab.

According to a preferred embodiment of the invention, the measurement of the expression of the gene encoding synoviolin is carried out in the following way:
a) biological material is extracted from the biological sample;
b) the biological material is brought into contact with at least one specific reagent for the gene encoding synoviolin;
c) the expression of the gene encoding synoviolin is determined.

For the purpose of the present invention, the term “biological sample” is intended to mean any sample taken from a patient, and liable to contain a biological material as defined hereinafter. This biological sample may in particular be a blood sample, serum sample, tissue sample or sample of synoviocytes from the patient. This biological sample is provided by any means of taking a sample known to those skilled in the art. According to a preferred embodiment of the invention, the biological sample taken from the patient is a blood sample.

In step a) of the method according to the invention, the biological material is extracted from the biological sample by any of the protocols for extracting and purifying nucleic acids known to those skilled in the art. For the purpose of the present invention, the term “biological material” is intended to mean any material that makes it possible to detect the expression of a target gene. The biological material may comprise in particular proteins, or nucleic acids such as, in particular, deoxyribonucleic acids (DNA) or ribonucleic acids (RNA). The nucleic acid may in particular be an RNA (ribonucleic acid). According to a preferred embodiment of the invention, the biological material extracted in step a) comprises nucleic acids, preferably RNA, and even more preferably total RNA. The total RNA comprises transfer RNAs, messenger RNAs (mRNAs), such as mRNAs transcribed from the target gene, but also transcribed from any other gene, and ribosomal RNAs. This biological material comprises material specific for a target gene, such as, in particular, the mRNAs transcribed from the target gene or the proteins derived from these mRNAs, but may also comprise material not specific for a target gene, such as, in particular, the mRNAs transcribed from a gene other than the target gene, the tRNAs, or rRNAs derived from genes other than the target gene.

By way of indication, the nucleic acid extraction can be carried out by means of:

a step consisting of lysis of the cells present in the biological sample, in order to release the nucleic acids contained in the patient's cells. By way of example, the lysis methods as described in patent applications WO 00/05338, WO 99/53304 and WO 99/15321 may be used. Those skilled in the art may use other well-known methods of lysis, such as thermal or osmotic shocks or chemical lyses using chaotropic agents such as guanidium salts (U.S. Pat. No. 5,234,809);

a purification step for separating the nucleic acids from the other cell constituents released in the lysis step. This step generally makes it possible to concentrate the nucleic acids, and can be adapted to the purification of DNA or RNA. By way of example, use may be made of magnetic particles optionally coated with oligonucleotides, by adsorption or covalence (in this respect, see patents U.S. Pat. No. 4,672,040 and U.S. Pat. No. 5,750,338), and the nucleic acids which are bound to these magnetic particles can be purified by means of a washing step. This nucleic acid purification step is particularly advantageous if it is desired to subsequently amplify said nucleic acids. A particularly advantageous embodiment of these magnetic particles is described in patent applications: WO-A-97/45202 and WO-A-99/35500. Another advantageous example of a nucleic acid purification method is the use of silica, either in the form of a column, or in the form of inert or magnetic particles. Other very widely used methods are based on ion exchange resins in a column or in a paramagnetic particulate format. Another method that is very relevant, but not exclusive, for the invention is that of adsorption onto a metal oxide substrate.

In step b), and for the purpose of the present invention, the term “specific reagent” is intended to mean a reagent which, when it is brought into contact with biological material as defined above, binds with the material specific for said target gene. By way of indication, when the specific reagent and the biological material are of nucleic origin, bringing the specific reagent and the biological material into contact allows hybridization of the specific reagent with the material specific for the target gene. The term “hybridization” is intended to mean the process during which, under suitable conditions, two nucleotide fragments bind to one another with stable and specific hydrogen bonds, so as to form a double-stranded complex. These hydrogen bonds form between the complementary bases adenine (A) and thymine (T) (or uracil (U)) (this is described as an A-T bond) or between the complementary bases guanine (G) and cytosine (C) (this is described as a G-C bond). The hybridization of two nucleotide fragments may be complete (reference is then made to complementary sequences or nucleotide fragments), i.e. the double-stranded complex obtained during this hybridization comprises only A-T bonds and C-G bonds. This hybridization may be partial (reference is then made to sufficiently complementary sequences or nucleotide fragments), i.e. the double-stranded complex obtained comprises A-T bonds and C-G bonds that make it possible to form the double-stranded complex, but also bases that are not bound to a complementary base. The hybridization between two nucleotide fragments depends on the operating conditions that are used, and in particular on the stringency. The stringency is defined in particular according to the base composition of the two nucleotide fragments, and also by the degree of mismatching between two nucleotide fragments. The stringency may also depend on the reaction parameters, such as the concentration and the type of ionic species present in the hybridization solution, the nature and the concentration of denaturing agents and/or the hybridization temperature. All these data are well known and the appropriate conditions can be determined by those skilled in the art. In general, depending on the length of the nucleotide fragments that it is desired to hybridize, the hybridization temperature is between approximately 20 and 70° C., in particular between 35 and 65° C. in a saline solution at a concentration of approximately 0.5 to 1 M. A sequence, or nucleotide fragment, or oligonucleotide, or polynucleotide, is a series of nucleotide motifs assembled together via phosphoric ester bonds, characterized by the informational sequence of the natural nucleic acids capable of hybridizing to a nucleotide fragment, it being possible for the series to contain monomers with different structures and to be obtained from a natural nucleic acid molecule and/or by genetic recombination and/or by chemical synthesis. A motif is derived from a monomer which may be a natural nucleotide of a nucleic acid, the constitutive elements of which are a sugar, a phosphate group and a nitrogenous base: in DNA, the sugar is deoxy-2-ribose, in RNA, the sugar is ribose; depending on whether DNA or RNA is involved, the nitrogenous base is chosen from adenine, guanine, uracil, cytosine and thymine; alternatively the monomer is a nucleotide modified in at least one of the three constitutive elements; by way of example, the modification may occur either at the level of the bases, with modified bases such as inosine, methyl-5-deoxycytidine, deoxyuridine, dimethylamino-5-deoxyuridine, diamino-2,6-purine, bromo-5-deoxyuridine or any other modified base capable of hybridization, either at the level of the sugar, for example the replacement of at least one deoxyribose with a polyamide, or else at the level of the phosphate group, for example replacement of the latter with esters chosen in particular from diphosphates, alkyl phosphonates, aryl phosphonates and phosphorothioates.

According to a specific embodiment of the invention, the specific reagent comprises at least one amplification primer. For the purposes of the present invention, the term “amplification primer” is intended to mean a nucleotide fragment comprising from 5 to 100 nucleic motifs, preferably from 15 to 30 nucleic motifs, for initiating an enzymatic polymerization, such as in particular an enzymatic amplification reaction. Preferably, a primer comprising all or part of a sequence of SEQ ID No. 1 or 2, preferably a pair of primers comprising SEQ ID No. 1 and SEQ ID No. 2 is used. The term “enzymatic amplification reaction” is intended to mean a process that generates multiple copies of a nucleotide fragment through the action of at least one enzyme. Such amplification reactions are well known to those skilled in the art and mention may in particular be made of the following techniques:

PCR (Polymerase Chain Reaction), as described in patents U.S. Pat. No. 4,683,195, U.S. Pat. No. 4,683,202 and U.S. Pat. No. 4,800,159,

LCR (Ligase Chain Reaction), disclosed, for example, in patent application EP 0 201 184,

RCR (Repair Chain Reaction), described in patent application WO 90/01069,

3SR (Self Sustained Sequence Replication) with patent application WO 90/06995,

NASBA (Nucleic Acid Sequence-Based Amplification) with patent application WO 91/02818, and

TMA (Transcription Mediated Amplification) with patent U.S. Pat. No. 5,399,491.

When the enzymatic amplification is a PCR, the specific reagent comprises at least 2 amplification primers, specific for a target gene, so as to make it possible to amplify the material specific for the target gene. Preferably, a primer comprising all or part of a sequence of SEQ ID No. 1 or 2 is used. The material specific for the target gene then preferably comprises a complementary DNA obtained by reverse transcription of messenger RNA derived from the target gene (reference is then made to target-gene-specific cDNA) or a complementary RNA obtained by transcription of the target-gene-specific cDNAs (reference is then made to target-gene-specific cRNA). When the enzymatic amplification is a PCR carried out after a reverse transcription reaction, this is then called an RT-PCR.

According to another preferred embodiment of the invention, the specific reagent of step b) comprises at least one hybridization probe.

The term “hybridization probe” is intended to mean a nucleotide fragment comprising at least five nucleotide motifs, such as from 5 to 100 nucleic motifs, in particular from 10 to 35 nucleic motifs, having a hybridization specificity under given conditions so as to form a hybridization complex with the material specific for a target gene. In the present invention, the material specific for the target gene may be a nucleotide sequence included in a messenger RNA derived from the target gene (reference is then made to a target-gene-specific mRNA), a nucleotide sequence included in a complementary DNA obtained by reverse transcription of said messenger RNA (reference is then made to a target-gene-specific cDNA), or else a nucleotide sequence included in a complementary RNA obtained by transcription of said cDNA as described above (reference will then be made to a target-gene-specific cRNA). The hybridization probe may comprise a label for the detection of said probe. The term “detection” is intended to mean either a direct detection by a physical method, or an indirect detection by a method of detection using a label. Many methods of detection exist for detecting nucleic acids [see, for example, Kricka et al., Clinical Chemistry, 1999, No. 45(4), p. 453-458 or Keller G. H. et al., DNA Probes, 2nd Ed., Stockton Press, 1993, sections 5 and 6, p. 173-249]. The term “label” is intended to mean a tracer capable of engendering a signal that can be detected. A nonlimiting list of these tracers comprises enzymes that produce a signal detectable, for example, by colorimetry, fluorescence or luminescence, such as horseradish peroxidase, alkaline phosphatase, beta-galactosidase, or glucose-6-phosphate dehydrogenase; chromophores such as fluorescent, luminescent or dye compounds; electron dense groups that can be detected by electron microscopy or by virtue of their electrical properties such as conductivity, by amperometry or voltammetry methods, or by impedance measurements; groups that can be detected by optical methods such as diffraction, surface plasmon resonance or contact angle variation, or by physical methods such as atomic force spectroscopy, tunnel effect, etc.; radioactive molecules such as ³²P, ³⁵S or ¹²⁵I.

For the purpose of the present invention, the hybridization probe may be a probe referred to as “detection probe”. In this case, the “detection” probe is labeled by means of a label as defined above. The detection probe can in particular be a “molecular beacon” detection probe as described by Tyagi & Kramer (Nature biotech, 1996, 14 :303-308). These “molecular beacons” become fluorescent during the hybridization. They have a stem-loop-type structure and contain a fluorophore and a “quencher” group. The binding of the specific loop sequence with its complementary target nucleic acid sequence causes the stem to unroll and the emission of a fluorescent signal during excitation at the appropriate wavelength.

For the detection of the hybridization reaction, use may be made of target sequences that have been labeled, directly (in particular by the incorporation of a label within the target sequence) or indirectly (in particular using a detection probe as defined above). It is in particular possible to carry out, before the hybridization step, a step consisting in labeling and/or cleaving the target sequence, for example using a labeled deoxyribonucleotide triphosphate during the enzymatic amplification reaction. The cleavage may be carried out in particular by the action of imidazole or of manganese chloride. The target sequence may also be labeled after the amplification step, for example by hybridizing a detection probe according to the sandwich hybridization technique described in document WO 91/19812. Another specific preferred method of labeling nucleic acids is described in application FR 2 780 059.

According to a preferred embodiment of the invention, the detection probe comprises a fluorophore and a quencher.

The hybridization probe may also be a probe referred to as a “capture probe”. In this case, the “capture” probe is immobilized or can be immobilized on a solid substrate by any appropriate means, i.e. directly or indirectly, for example by covalence or adsorption. As solid substrate, use may be made of synthetic materials or natural materials, optionally chemically modified, in particular polysaccharides such as cellulose-based materials, for example paper, cellulose derivatives such as cellulose acetate and nitrocellulose or dextran, polymers, copolymers, in particular based on styrene-type monomers, natural fibers such as cotton, and synthetic fibers such as nylon; inorganic materials such as silica, quartz, glasses or ceramics; latices; magnetic particles; metal derivatives, gels, etc. The solid substrate may be in the form of a microtitration plate, of a membrane as described in application WO-A-94/12670, or of a particle. These steps of hybridization on a substrate may be preceded by an enzymatic amplification reaction step, as defined above, in order to increase the amount of target genetic material.

In step c), the determination of the expression of the target gene can be carried out by any of the protocols known to those skilled in the art.

In general, the expression of a target gene can be analyzed by detecting the mRNAs (messenger RNAs) that are transcribed from the target gene at a given moment or by detecting the proteins derived from these mRNAs.

The invention preferably relates to the determination of the expression of a target gene by detection of the mRNAs derived from this target gene.

When the specific reagent comprises one or more amplification primers, it is possible, in step c) of the method according to the invention, to determine the expression of the target gene in the following way:

1) After having extracted, as biological material, the total RNA (comprising the transfer RNAs (tRNAs), the ribosomal RNAs (rRNAs) and the messenger RNAs (mRNAs)) from a biological sample as presented above, a reverse transcription step is carried out in order to obtain the complementary DNAs (or cDNAs) of said mRNAs. By way of indication, this reverse transcription reaction can be carried out using a reverse transcriptase enzyme which makes it possible to obtain, from an RNA fragment, a complementary DNA fragment. The reverse transcriptase enzyme from AMV (Avian Myoblastosis Virus) or from MMLV (Moloney Murine Leukaemia Virus) can in particular be used. When it is more particularly desired to obtain only the cDNAs of the mRNAs, this reverse transcription step is carried out in the presence of nucleotide fragments comprising only thymine bases (polyT), which hybridize by complementarity to the polyA sequence of the mRNAs so as to form a polyT-polyA complex which then serves as a starting point for the reverse transcription reaction carried out by the reverse transcriptase enzyme. cDNAs complementary to the mRNAs derived from a target gene (target-gene-specific cDNA) and cDNAs complementary to the mRNAs derived from genes other than the target gene (cDNAs not specific for the target gene) are then obtained.

2) The amplification primer(s) specific for a target gene is (are) brought into contact with the target-gene-specific cDNAs and the cDNAs not specific for the target gene. The amplification primer(s) specific for a target gene hybridize(s) with the target-gene-specific cDNAs and a predetermined region, of known length, of the cDNAs originating from the mRNAs derived from the target gene is specifically amplified. The cDNAs not specific for the target gene are not amplified, whereas a large amount of target-gene-specific cDNAs is then obtained. For the purpose of the present invention, reference is made, without distinction, to “target-gene-specific cDNAs” or to “cDNAs originating from the mRNAs derived from the target gene”. This step can be carried out in particular by means of a PCR-type amplification reaction or by any other amplification technique as defined above.

3) The expression of the target gene is determined by detecting and quantifying the target-gene-specific cDNAs obtained in step 2) above. This detection can be carried out after electrophoretic migration of the target-gene-specific cDNAs according to their size. The gel and the medium for the migration can include ethidium bromide so as to allow direct detection of the target-gene-specific cDNAs when the gel is placed, after a given migration period, on a UV (ultraviolet)-ray light table, through the emission of a light signal. The greater the amount of target-gene-specific cDNAs, the brighter this light signal. These electrophoresis techniques are well known to those skilled in the art. The target-gene-specific cDNAs can also be detected and quantified using a quantification range obtained by means of an amplification reaction carried out until saturation. In order to take into account the variability in enzymatic efficiency that may be observed during the various steps (reverse transcription, PCR, etc.), the expression of a target gene of various groups of patients can be normalized by simultaneously determining the expression of a “housekeeping” gene, the expression of which is similar in the various groups of patients. By realizing a ratio of the expression of the target gene to the expression of the housekeeping gene, i.e. by realizing a ratio of the amount of target-gene-specific cDNAs to the amount of housekeeping-gene-specific cDNAs, any variability between the various experiments is thus corrected. Those skilled in the art may refer in particular to the following publications: Bustin S A, J Mol Endocrinol, 2002, 29: 23-39; Giulietti A Methods, 2001, 25: 386-401.

When the specific reagent comprises at least one hybridization probe, the expression of a target gene can be determined in the following way:

1) After having extracted, as biological material, the total RNA from a biological sample as presented above, a reverse transcription step is carried out as described above in order to obtain cDNAs complementary to the mRNAs derived from a target gene (target-gene-specific cDNA) and cDNAs complementary to the mRNAs derived from genes other than the target gene (cDNA not specific for the target gene).

2) All the cDNAs are brought into contact with a substrate, on which are immobilized capture probes specific for the target gene whose expression it is desired to analyze, in order to carry out a hybridization reaction between the target-gene-specific cDNAs and the capture probes, the cDNAs not specific for the target gene not hybridizing to the capture probes. The hybridization reaction can be carried out on a solid substrate which includes all the materials as indicated above. According to a preferred embodiment, the hybridization probe is immobilized on a substrate. The hybridization reaction may be preceded by a step consisting of enzymatic amplification of the target-gene-specific cDNAs as described above, so as to obtain a large amount of target-gene-specific cDNAs and to increase the probability of a target-gene-specific cDNA hybridizing to a capture probe specific for the target gene. The hybridization reaction may also be preceded by a step consisting in labeling and/or cleaving the target-gene-specific cDNAs as described above, for example using a labeled deoxyribonucleotide triphosphate for the amplification reaction. The cleavage can be carried out in particular by the action of imidazole and manganese chloride. The target-gene-specific cDNA can also be labeled after the amplification step, for example by hybridizing a labeled probe according to the sandwich hybridization technique described in document WO-A-91/19812. Other preferred specific methods for labeling and/or cleaving nucleic acids are described in applications WO 99/65926, WO 01/44507, WO 01/44506, WO 02/090584, WO 02/090319.

3) A step consisting of detection of the hybridization reaction is subsequently carried out. The detection can be carried out by bringing the substrate on which the capture probes specific for the target gene are hybridized with the target-gene-specific cDNAs into contact with a “detection” probe labeled with a label, and detecting the signal emitted by the label. When the target-gene-specific cDNA has been labeled beforehand with a label, the signal emitted by the label is detected directly.

When the at least one specific reagent brought into contact in step b) of the method according to the invention comprises at least one hybridization probe, the expression of a target gene can also be determined in the following way:

1) After having extracted, as biological material, the total RNA from a biological sample as presented above, a reverse transcription step is carried out as described above in order to obtain the cDNAs of the mRNAs of the biological material. The polymerization of the complementary RNA of the cDNA is subsequently carried out using a T7 polymerase enzyme which functions under the control of a promoter and which makes it possible to obtain, from a DNA template, the complementary RNA. The cRNAs of the cDNAs of the mRNAs specific for the target gene (reference is then made to target-gene-specific cRNA) and the cRNAs of the cDNAs of the mRNAs not specific for the target gene are then obtained.

2) All the cRNAs are brought into contact with a substrate on which are immobilized capture probes specific for the target gene whose expression it is desired to analyze, in order to carry out a hybridization reaction between the target-gene-specific cRNAs and the capture probes, the cRNAs not specific for the target gene not hybridizing on the capture probes. The hybridization reaction may also be preceded by a step consisting of labeling and/or cleavage of the target-gene-specific cRNAs as described above.

3) A step consisting in detecting the hybridization reaction is subsequently carried out. The detection can be carried out by bringing the substrate on which the capture probes specific for the target gene are hybridized with the target-gene-specific cRNA into contact with a “detection probe” labeled with a label, and detecting the signal emitted by the label. When the target-gene-specific cRNA has been labeled beforehand with a label, the signal emitted by the label is detected directly. The use of cRNA is particularly advantageous when a substrate of biochip type on which a large number of probes are hybridized is used.

According to a specific embodiment of the invention, steps B and C are carried out at the same time. This preferred method can in particular be carried out by “real time NASBA”, which combines, in a single step, the NASBA amplification technique and real-time detection which uses “molecular beacons”. The NASBA reaction takes place in the tube, producing the single-stranded RNA with which the specific “molecular beacons” can simultaneously hybridize to give a fluorescent signal. The formation of the new RNA molecules is measured in real time by continuous verification of the signal in a fluorescent reader.

The invention also relates to the use of at least one specific reagent for the gene encoding synoviolin, as defined above, for determining the response of a patient suffering from rheumatoid arthritis to a treatment directed against a cytokine involved in the inflammatory process of the disease or for monitoring the response of a patient suffering from rheumatoid arthritis to a treatment directed against a cytokine involved in the inflammatory process of the disease, over time. Preferably, the treatment is directed against the TNF-alpha cytokine. In this respect, mention may be made of a treatment that blocks the action of TNF, such as, in particular, Infliximab®, etanercept and adalimumab.

Finally, the invention relates to a kit for the prognosis of the response of a patient suffering from rheumatoid arthritis to a treatment directed against a cytokine involved in the inflammatory process of the disease, said treatment being as defined above, comprising at least one specific reagent for the gene encoding synoviolin, as defined above.

The invention also relates to a kit for monitoring the response of a patient suffering from rheumatoid arthritis to a treatment directed against a cytokine involved in the inflammatory process of the disease, said treatment being as defined above, comprising at least one specific reagent for the gene encoding synoviolin, as defined above.

The analysis of the expression of synoviolin then makes it possible to provide a tool for the prognosis of the response of a patient suffering from rheumatoid arthritis to a treatment directed against cytokine involved in the inflammatory process of the disease. It is possible, for example, to analyze the expression of the target gene in a patient whose reaction to a treatment directed against a cytokine involved in the inflammatory process of the disease is unknown, and to compare with known average expression values for the target gene of patients who respond to said treatment and known average expression values for the target gene of patients who do not respond to said treatment. This makes it possible to determine whether the patient is a responder or a nonresponder, thereby making it possible to provide said patient with an appropriate treatment or to adapt said patient's treatment throughout his or her therapy.

The following example is given by way of illustration and is in no way limiting in nature. It will make it possible to understand the invention more clearly.

### EXAMPLE

### Study of the Expression of the Gene Encoding Synoviolin for the Diagnosis/Prognosis of Rheumatoid Arthritis

Patients—The study was carried out on control patients (C, n=23) or patients suffering from rheumatoid arthritis (RA, n=47). The RA and C groups of patients had a similar sex ratio and average age. The RA patients were categorized according to the revised criteria of the American College of Rheumatology (Arnett et al. Arthirtis Rheum 1988; 31:315-324). The blood samples were taken and collected in PAXGene™ Blood RNA tubes (PreAnalytix, Hilden, Germany).

Treatment—All the patients suffering from rheumatoid arthritis received an intravenous injection of 3 mg/kg of Infliximab® on weeks 0, 2, 6, 14, and 22. Infliximab® is a TNF-alpha inhibitor. TNF-alpha inhibitors produce a rapid improvement in the clinical and biological signs in rheumatoid arthritis that is refractory to other treatments, in particular to methotrexate. Infliximab® or remicade® is a chimeric anti-TNF-alpha antibody. A methotrexate (MTX) treatment was also prescribed. The clinical response was assessed after the first injection (week 0) and immediately after the fifth injection (week 22) using the following criteria: joint pain, joint swelling, patient pain assessment, overall assessment of the disease from the patient's point of view, overall assessment of the disease from the physician's point of view, a Health Assessment Questionnaire, the C-reactive protein (CRP) serum level and the erythrocyte sedimentation rate (ESR). Patients who were good responders (GR) to the treatment and who were poor responders (PR) to the treatment were distinguished.

Synoviocyte culture—A fibroblast-like synoviocyte (FLS) cell line was obtained from a sample from patients (RA). Control cells derived from a skin sample from RA patients were also analyzed (CT). The FLS and CT were isolated with a digesting enzyme and placed in culture in an RPMI medium comprising 10% of fetal calf serum (Invitrogen) at 37° C. in a humid incubator comprising 5% of CO₂ as previously described (Toh et al. Arthritis Rheum 2004 October; 50(10):3118-3128). The synoviocytes, and also the control cells, were subsequently cultured in 96-well microplates (10 000 cells per well) in a final volume of 200 μl in culture medium supplemented with 10% FCS and treated with IL-1β (0.1 ng/ml, Immunotools, Friesoythe, Germany) or TNFα? (1 ng/ml, Immunotools, Friesoythe, Germany).

Analysis of the expression of the gene encoding synoviolin—The expression of the gene encoding synoviolin was measured by real-time PCR in the peripheral blood of control patients (C) and of patients suffering from rheumatoid arthritis (RA), before and after 6 months of treatment with Infliximab®. The expression of the gene encoding synoviolin was also measured in the FLS cell line and the CT control cells. The gene encoding cyclophilin B (PPIP) was used as a housekeeping gene for the blood samples, and the gene encoding beta-actin was used as a housekeeping gene for the FLS cell lines and the CT control cells as previously described (Pachot et al, J. Biotechnology 2004; 114:121-124; Toh et al, Arthritis Rheum 2004; 50:3118-3128.). The total RNA was extracted from whole blood using the PAXGene™ Blood RNA kit (PreAnalytix) and was purified using an RNA-easy kit (Qiagen, Hilden, Germany). The RNA was extracted from the FLS and CT cells using TRIzol (Gibco BRL) and was purified using an RNA-easy kit (Qiagen, Hilden, Germany). The cDNAs were prepared from 1 μg of total RNA using a Thermoscript RT-PCR system (Invitrogen, California, USA). An amount of 1 μg of RNA was reverse transcribed using the Thermoscript RT-PCR system (Invitrogen, California, USA) and a PCR amplification was carried out on a LightCycler (Roche) using the Fast-Start™ DNA Master SYBR Green I real-time PCR kit (Roche Molecular Biochemicals). The primers specific for synoviolin that were used were the following:

| | |
|---|---|
| SEQ ID No. 1: | 5′-GTT TAC AGG CTT CAT CAA GG-3′ |
| and | |
| SEQ ID No. 2: | 5′-CAT GAT GGC ATC TGT CAC AG-3′. |

For cyclophilin B, the primers were obtained from LC-Search (PPIB, accession number: M60857, amplicon 105 to 338). For beta-actin, the primers used were the following (Toh et al, Arthritis Rheum. 2004 October; 50(10):3118-3128):

| | |
|---|---|
| SEQ ID No. 3: | 5′-TGTCCCTGTATGCCTCTGGT-3′ |
| and | |
| SEQ ID No. 4 | ′-GATGTCACGCACGATTTCC-5′. |

A volume of 10 μl of standard cDNA and of cDNA dilutions originating from the samples was added to the capillary tubes. The amplification was carried out in a final volume of 20 μl comprising a primer concentration of 10 μM, 25 mM of magnesium chloride (MgCl₂), and also the Taq enzyme and the SYBR Green I label contained in the LightCycler Fast start DNA Master SYBR green I kit (Roche). The PCR was carried out for 45 amplification cycles (10 seconds at 95° C., 10 seconds at 68° C. and 16 seconds at 72° C.). For each of the genes of interest, a standard was prepared by means of a PCR (polymerase chain reaction) amplification carried out until saturation. The amplicons obtained were purified (PCR purification kit, Qiagen Ltd) and the presence of a single amplicon was verified by agarose gel electrophoresis and ethidium bromide staining. The mRNA expression was determined using the LightCycler software.

Statistical analyses—The results were expressed by the mean±SEM. The differences in values were calculated using an ANOVA test, and the values having a probability of less than 0.05 were considered to be significantly different.

Results

Analysis of the Expression of Synoviolin in Control Patients and Patients Suffering from Rheumatoid Arthritis

The results obtained are given in table 1 below.

TABLE 1

| | C patients | RA patients | p |
|---|---|---|---|
| Synoviolin mRNA | 0.31 ± 0.10 | 0.70 ± 0.46 | <0.001 |
| expression | | | |

This table 1 gives the level of expression of the mRNAs of the gene encoding synoviolin in control patients (C) and patients suffering from rheumatoid arthritis (RA). The results are expressed in terms of the ratio of relative quantification between the mRNAs of the target gene and the mRNAs of the cyclophilin B housekeeping gene. The results are expressed in terms of the mean of the ratios obtained for each of the groups of patients. The SEM (standard error of the mean) was also calculated for each of the groups. The values given in table 1 are the means obtained ± the SEM. The values were considered to be statistically different when the value of p obtained was less than 0.05.

The RA patients showed a synoviolin mRNA expression level that was significantly increased compared with the control patients.

### Analysis of the Expression of Synoviolin in the FLS Cell Lines and the CT Control Cells

The results obtained are given in tables 3 and 4 below.

TABLE 2

| Hour following the addition | Ratio of synoviolin/ | Ratio of synoviolin/ |
|---|---|---|
| of IL1β or of TNFα to | β-actin in the | β-actin in the |
| the culture medium | presence of Il-1β | presence of TNFα |
| 0 | 1 | 1 |
| 3 | 1.27 ± 0.13 | 1.02 ± 0.02 |
| 6 | 3.75 ± 0.84 | 2.27 ± 0.73 |
| 24 | 4.05 ± 0.80 | 1.33 ± 0.24 |

Table 2 gives the ratio of relative quantification between the mRNAs of the target gene and the mRNAs of the beta-actin housekeeping gene in FLS cells cultured in the presence of IL1-beta (0.1 ng/ml) or of TNF-alpha (1 ng/ml). TNF increased the expression of synoviolin for 6 h. IL1 also increased the expression of synoviolin, which remained high up to 24 h.

TABLE 3

| [ ] of IL-1β | synoviolin/β-actin mRNA | synoviolin/b-actin mRNA |
|---|---|---|
| or TNFα | in the presence of | in the presence of |
| (ng/ml) | various [ ] of IL-1β | various [ ] of TNFα |
| 0 | 14.455 ± 3.36 | 14.46 ± 3.36 |
| 0.1 | 92.71 ± 21.57 | |
| 1 | 156.93 ± 36.52 | 66.29 ± 15.43 |
| 10 | 143.46 ± 33.38 | 70.44 ± 16.39 |

Table 3 gives the ratio of relative quantification between the mRNAs of the target gene and the mRNAs of the beta-actin housekeeping gene in FLS cells cultured in the presence of various concentrations of IL1-beta or of TNF-alpha. The ratio was dependent on the concentration of IL1-beta and TNF-alpha.

These results demonstrate that the upregulation of synoviolin by TNF-alpha and IL1-beta can contribute to the disruption of FLS proliferation in rheumatoid arthritis.

TABLE 4

| Hour following the addition | Ratio of synoviolin/ | Ratio of synoviolin/ |
|---|---|---|
| of IL1β or of TNFα to | β-actin in the | β-actin in the |
| the culture medium | presence of Il-1b | presence of TNFα |
| 0 | 1 | 1 |
| 6 | 2.31 ± 0.80 | 2.04 ± 0.97 |

Table 4 gives the ratio of relative quantification between the mRNAs of the target gene and the mRNAs of the beta-actin housekeeping gene in CT control cells cultured in the presence of IL1-beta (0.1 ng/ml) or of TNF-alpha (1 ng/ml). No significant increase in synoviolin expression was observed in the presence of IL1 or of TNF.

Analysis of the Expression of Synoviolin in Patients Suffering from Rheumatoid Arthritis who Respond to an Infliximab® Treatment and in Patients Suffering from Rheumatoid Arthritis who do not Respond to an Infliximab® Treatment

The results obtained are given in table 5 below.

TABLE 5

| | | GR patients | PR patients | P |
|---|---|---|---|---|
| Synoviolin mRNA | Before | 0.55 ± 0.27 | 1.25 ± 0.60 | <0.005 |
| expression | treatment | | | |
| | After | 0.32 ± 0.13 | 0.95 ± 0.63 | <0.005 |
| | treatment | | | |
| | P | <0.005 | NS | |

This table 5 gives the level of expression of the mRNAs of the gene encoding synoviolin in patients who respond (GR) and patients who do not respond (PR), before and 6 months after treatment. The results are expressed in terms of the ratio of relative quantification between the mRNAs of the target gene and the mRNAs of the cyclophilin B housekeeping gene. The results are expressed in terms of the mean of the ratios obtained for each of the groups of patients. The SEM (standard error of the mean) was also calculated for each of the groups. The values given in table 5 are the means obtained ± the SEM. The PR patients showed a synoviolin mRNA expression level that was significantly increased before and after the treatment, compared with the GP patients. The expression of synoviolin mRNAs was decreased after 6 months of treatment in GP patients but not in the PR patients. Synoviolin expression was significantly increased in the GP patients compared with the control patients.

Conclusion—The study of the expression of the mRNAs of the genes encoding synoviolin using samples of whole blood thus makes it possible to very effectively discriminate between the patients suffering from rheumatoid arthritis and the control patients, but also makes it possible to discriminate between the patients who are good responders to an Infliximab® treatment and patients who are poor responders to an Infliximab® treatment. Synoviolin is therefore an excellent marker for the diagnosis of rheumatoid arthritis, but especially for the prognosis as regards the response of a patient with respect to a treatment. Synoviolin is also an excellent marker for monitoring a patient being treated with Infliximab®. This allows the physician to rapidly identify patients who do not respond to Infliximab®, thereby making it possible to give them another treatment that is more suitable.

1. An in vitro method for determining, from a biological sample, the response of a patient suffering from rheumatoid arthritis to a treatment directed against a cytokine involved in the inflammatory process of the disease or for monitoring the response of a patient suffering from rheumatoid arthritis to a treatment directed against a cytokine involved in the inflammatory process of the disease, over time, characterized in that the expression of the gene encoding synoviolin is measured.

2. The method as claimed in claim 1, in which the measurement of the expression of the gene encoding synoviolin comprises the following steps:
a. biological material is extracted from the biological sample,
b. the biological material is brought into contact with at least one specific reagent for the gene encoding synoviolin;
c. the expression of the gene encoding synoviolin is determined.

3. The method as claimed in claim 2, characterized in that the biological material extracted in step a) comprises nucleic acids.

4. The method as claimed in claim 2, characterized in that the at least one specific reagent of step b) comprises at least one hybridization probe.

5. A method for the gene encoding synoviolin, for determining the response of a patient suffering from rheumatoid arthritis to a treatment directed against a cytokine involved in the inflammatory process of the disease or for monitoring the response of a patient suffering from rheumatoid arthritis to a treatment directed against a cytokine involved in the inflammatory process of the disease, over time utilizing at least one specific reagent.

6. A kit for the prognosis of the response of a patient suffering from rheumatoid arthritis to a treatment directed against a cytokine involved in the inflammatory process of the disease, comprising at least one specific reagent for the gene encoding synoviolin.

7. A kit for monitoring the response of a patient suffering from rheumatoid arthritis to a treatment directed against a cytokine involved in the inflammatory process of the disease, comprising at least one specific reagent for the gene encoding synoviolin, as defined above.
